(19) United States
(12) Patent Application Publication
(10) Pub. No.: US 2014/0278130 A1
(43) Pub. Date: Sep. 18, 2014

(54) Method of predicting toxicity for chemical compounds

(71) Applicants: William Michael Bowles, San Jose, CA (US); Ronald T. Shigeta, JR., Berkeley, CA (US)

(72) Inventors: William Michael Bowles, San Jose, CA (US); Ronald T. Shigeta, JR., Berkeley, CA (US)

(21) Appl. No.: 13/999,615

(22) Filed: Mar. 12, 2014

Related U.S. Application Data
(60) Provisional application No. 61/852,322, filed on Mar. 14, 2013.

(51) Int. Cl. G06F 19/00; G01N 33/15

(52) U.S. Cl. CPC G06F 19/704; G01N 33/15
USPC 702/19

(57) ABSTRACT

The invention disclosed herewith is a computer-implemented method for evaluating the toxicity of chemical compounds. In particular, some embodiments of the invention comprise importing microarray data representing measurements of the RNA transcription from hepatocytes, and running at least one algorithm (such as a coefficient penalized linear regression algorithm) on the imported data to assess potential adverse drug effects. After the evaluation has been carried out, the results are exported to reports or databases.

In some embodiments of the invention, the algorithm has been trained on reference data using machine learning techniques.

In some embodiments of the invention, the evaluation of toxicity is carried out concurrently with the evaluation of efficacy, where it can be used to assess the clinical value of the compounds evaluated.

In some embodiments of the invention, the evaluation of toxicity is inserted into a pharmaceutical evaluation process prior to expensive testing of toxicity in animals.

### CROSS-REFERENCE TO RELATED APPLICATIONS

This patent application claims the benefit of U.S. Provisional Patent Application No. 61/852,322, filed on Mar. 13, 2013, which is also incorporated herein by reference in its entirety.

### FIELD OF THE INVENTION

This invention relates to the field of testing potential pharmaceutical molecules and compounds for toxicity. More specifically, it relates to making a numerical evaluation by a computer of data collected about candidate chemical compounds to predict the potential toxicity of those compounds. These toxicity predictions can then be used to guide subsequent pharmaceutical testing protocols, such as whether preclinical trials using animal testing should be conducted.

### BACKGROUND TO THE INVENTION

### The Drug Development Process.

The protocols in the development of new chemical compounds for use pharmaceuticals have undergone a dramatic transformation in recent decades. Instead of simply attempting to synthesize a number of chemical compounds found in nature and then testing them in animals, the development of procedures using high throughput screens (HTS) (for example, using microtiter plates) allows screening for potential efficacy of new compounds for thousands of chemical structures in parallel [See, for example X. D. Zhang, Optimal High-Throughput Screening: Practical Experimental Design and Data Analysis for Genome-scale RNAi Research. (Cambridge University Press, New York, N.Y., 2011)].

FIG. 1 illustrates the typical steps of a modern drug development protocol. The illustration and following description are adapted from data presented in the article by Steven M. Paul et al., entitled “How to improve R&D productivity: the pharmaceutical industry's grand challenge” [Nature Reviews, vol. 9, pp. 203-214 (March 2010)].

In the first step, the “Target-to-Hit” step 010, new candidate compounds, also known as candidate molecular entities (CMEs) 009, are evaluated using high throughput screening procedures. In this application, the term “candidate molecular entities” or CMEs, will be used to represent a number of entities with the potential to become drugs, including: newly invented molecular entities (sometimes called “new molecular entities”, or NMEs); known molecules that may have been tested before but have never been approved for use as drugs; known molecules that are already used as drugs in various therapeutical treatments, but that may be retargeted for new or different therapeutical uses; molecules that have been approved as drugs and are included as a known compound, or as a control on the experimental methodology; and the like. CMEs may also include combinations of molecules, some expected to be potential drugs, and others provided as nominally inert host or buffer materials, but which may, in various combinations, affect the efficacy of the potential drug.

The compounds that that trigger certain responses in an HTS process are called “screening hits”. Once identified, in the next step, the “Hit-to-Lead” step 020, additional compounds similar to the “hits” are synthesized, and again run through HTS experiments. The variations can often show improvements in response by several orders of magnitude. A molecule with a suitably large response is called a “lead”.

In the third step, the “Lead Optimization” step 030, further variants and formulations of the lead compounds can then be further tested and optimized, until one appears promising enough to warrant testing in animals and then humans.

Up until this point, most of these experiments involve testing many CMEs in parallel, with screening tests often involving hundreds or even thousands of CMEs. Once a lead has been identified, the next steps tend to be trials focused on a small number, or even a single, molecule, conducted with the goal of determining efficacy, toxicity, and the details of therapeutic treatment (such as dose) for that identified lead.

In the next step, the Preclinical Trials step 040, the lead compound is tested using in vitro (test tube or cell culture) experiments and in vivo (animal) experiments using wide-ranging doses to obtain preliminary efficacy, toxicity and pharmacokinetic information. The main goal of a pre-clinical trial study is to determine a potential drug's ultimate safety profile—if animals die or show serious side effects, the development of that CME as a potential drug will stop.

In the next steps, the Phase I Clinical Trials step 050, the Phase II Clinical Trials step 060, and the Phase III Clinical Trials step 070, testing in humans is carried out. Phase I Trials 050 typically test a candidate compound in 10 to 100 healthy humans, generally determining safety, identifying side effects, and also establishing dosing protocols. If no significant ill effects are identified, Phase II Trials 060 then test the candidate compound in 100-300 patients, to establish efficacy in treating a human medical condition. If the new compound is found to be effective, Phase III Trials 070 are conducted using the candidate compound in 1,000-2,000 patients, to determine the therapeutic effect and also establish the value as a medical treatment. In each of these trials, adverse results may cause a CME to be discontinued as a potential drug.

In the final step, the Submission step 080, the data from the various clinical trials are gathered together and submitted to an agency such as the U.S. Food and Drug Administration for approval as a medical treatment. Once approved, the pharmaceutical company can begin manufacturing and marketing drugs 090 using the CME.

FIG. 1 also shows the number of molecules, on average, that pass each step in the protocol. For each one (1) CME that is introduced to the market as a drug 090, at least twenty-four (24) CMEs entered the process, shown symbolically with 24 molecules 009 in the initial “Target-to-Hit” step 010. Typical counts for the number of the 24 initial CMEs that pass a given step in the protocol (as presented in the above cited article by Paul) are shown in the bottom left corner of each rectangle representing a step in the protocol. The cost of evaluating each CME for that step in the protocol is shown in at the right side of each rectangle.

As an example of the use of these numbers from FIG. 1, for the Pre-Clinical (animal) Trials 040, fifteen (15) compounds may have been identified as “leads”, in the previous Lead Optimization step 030, and each will require a Pre-Clinical Trial 040. Each pre-clinical trial for each identified lead will cost on average $5M each, for a total cost of 15×$5M=$75M. Of these fifteen (15) compounds, according to the data presented in the article by Paul, on average three (3) compounds would be eliminated for various reasons (most likely toxicity results), and so only twelve (12) of the original fifteen (15) molecules would graduate to Phase I Trials 050.

Furthermore, for the Phase I Clinical Trials 050, each of these twelve (12) compounds is evaluated at a cost of $15M per molecule, for a total cost of 12×$15M=$180M. Again according to the data presented in the article by Paul, of these twelve (12) compounds, on average three (3) compounds would be eliminated for various reasons (most likely adverse side effects or other toxic effects), and so only nine (9) of the original twelve (12) molecules would graduate to Phase II Trials 060.

Finding some way to predict the toxicity of these compounds in advance can have great financial benefits. As illustrated in FIG. 2, a modified Lead Optimization step 032, for example, that could identify the toxicity of the six (6) compounds mentioned above could eliminate the cost of testing these six (6) failed CMEs in pre-clinical trials 040 (saving $5M per compound) as well as the cost of testing three (3) of these compounds in Phase I Trials 050 (saving $15M per compound), for a total savings of $75M.

Finding a way to predict both efficacy and toxicity in parallel may also yield unexpected benefits. Shown in FIG. 3 is a hypothetical example of the evaluation of five (5) CMEs 110, labeled A, B, C, D, and E. Assume that, for example, a compound must have both a high score S for efficacy (i.e. a score of 0 would mean no effect; a score of 100 would indicate an ideal outcome) from an efficacy evaluation 120, and a low score T on a toxicity scale (i.e. a score of 0 would indicate no toxic effects; a score of 100 would be lethal) from a toxicity evaluation 130. Consider the ratio of the two scores S/T as a figure of merit (FOM) for a CME.

Hypothetical results are shown in Table I. For these five (5) examples of CMEs, the best possible outcome is for CME B, which is fairly effective and also non-toxic. The worst outcome is for CME A, which is both ineffective has the side effect of being almost 100% lethal.

TABLE I
Hypothetical Figure of Merit (FOM) = S/T derived from Efficacy
Scores S and Toxicity Scores (T) for 5 hypothetical CMEs.

| Compound (CME) | S | T | FOM |
|---|---|---|---|
| A | 10 | 99 | 0.10 |
| B | 55 | 5 | 11.00 |
| C | 88 | 95 | 0.92 |
| D | 70 | 44 | 1.59 |
| E | 25 | 58 | 0.43 |

When these are evaluated sequentially, as is illustrated in FIG. 3, the top two (2) CMEs (CMEs C and D) appear significantly superior to the others, and a likely outcome of this screening would be to allow trials to proceed for only these two (2) compounds, allowing the remaining three (3) CMEs to be abandoned.

However, in the next step, the toxicity evaluation 130 (typically corresponding to the Pre-clinical Trials 040 of FIGS. 1 and 2), CME C turns out to be particularly lethal. Such a compound would be quickly abandoned, and the only surviving compound of the group would be CME D.

In contrast to this sequential approach, if there were an opportunity to conduct efficacy trials and the toxicity trials independently in parallel, as illustrated in FIG. 4, ideally as part of the Lead Optimization step 032, the toxic effects of CME C could be predicted, and the cost of the subsequent pre-clinical trials for CME C avoided. Likewise, the highly beneficial toxicity score for CME B, along with its FOM, can be determined, correctly identifying it as the most attractive candidate for further trials.

Until now, the use of high throughput screening (HTS) has mostly been for the identification of the positive efficacy for a drug. High throughput techniques to similarly evaluate toxic effects have not been well developed.

There have been some attempts to make predictions of toxicity results. There are known methods that use a preparation of hepatocyte cells, which are liver cells maintained in a culture medium [see P. Papeleu et al., “Isolation of Rat Hepatocytes,” in Methods in Molecular Biology, vol. 320: Cytochrome P450 Protocols, 2^{nd} Ed, I. Phillips & E. Shephard, eds., pp 229-237 (Humana Press, Totowa, N.J., 2005)]. Several studies attempting to predict various toxic effects using gene expression data have been carried out. [See, for example, M. R. Fielden et al., “Interlaboratory evaluation of genomic signatures for predicting carcinogenicity in the rat”, Toxicol. Sci. vol. 103(1), pp. 28-34 (2008); M. Chen et al., “A decade of toxicogenomic research and its contribution to toxicological science.” Toxicol. Sci. vol. 130, pp. 217-28 (2012); K. F. Johnson & S. M. Lin, “Call to work together on microarray data analysis.” Nature vol. 411, p. 885 (2001); and A. Y. Nie et al., “Predictive toxicogenomics approaches reveal underlying molecular mechanisms of nongenotoxic carcinogenicity”, Mol. Carcinog. vol. 45, pp. 914-33 (2006)].

“Chemical fingerprints” for a compound are also a prior art technique that has been applied to predict toxicity. The decomposition of the atomic and molecular structure for a chemical compound into a list of features (the “chemical fingerprint”) provides a convenient way to assess the similarity between chemical compounds and their potential biological or pharmaceutical activity. Examples of the algorithms used to determine the fingerprint of the structure of a chemical compound are methods have been disclosed [see A. Bender et al., “Similarity Searching of Chemical Databases Using Atom Environment Descriptors (MOLPRINT 2D): Evaluation of Performance,” J. Chem. Inf. Comput. Sci., 44(5), pp 1708-1718 (2004)].

There is, however, a need for a more systematic and comprehensive approach to predict toxicity results for candidate molecular entities (CMEs) based on statistically large volumes of collected data. Such predictive power may save hundreds of millions of dollars by eliminating toxic compounds from further evaluation.

### BRIEF SUMMARY OF THE INVENTION

The invention disclosed with this application is a method using computer machine learning techniques for evaluating the toxicity of a chemical compound. In particular, some embodiments of the invention comprise importing microarray data representing measurements for new molecular entities (CMEs) or other compounds of the RNA transcription from hepatocytes (cell lines derived from liver), and running at least one machine learning model (such as a coefficient penalized linear regression algorithm) to make an assessment of toxicity or other adverse effects. After determining the evaluation of toxicity, the results are exported to databases or as reports.

In some embodiments of the invention, the machine learning model may comprise multiple algorithms. In some embodiments of the invention, the machine learning model has been trained using a dataset of known toxicity results for known compounds, and the selection of algorithms and parameters to be used in the model identified prior to the application to data from CMEs.

In some embodiments of the invention, a machine learning model is used to predict potential toxicity for CMEs, and is inserted into a pharmaceutical protocol for those CMEs prior to expensive pre-clinical testing for toxicity in cells or animals.

In some embodiments of the invention, the evaluation of toxicity is carried out concurrently with evaluations of efficacy, where it can be used to assess the clinical value of the compounds evaluated.

In some embodiments of the invention, the evaluation of toxicity can be used for a preliminary assessment of the toxicity of a compound before efficacy trials have been attempted.

### BRIEF DESCRIPTION OF THE DRAWINGS

FIG. 1 illustrates the typical steps for a research protocol for drug discovery and evaluation, along with a representation of the probability of a compound becoming a drug.

FIG. 2 illustrates the typical steps for a research protocol for drug discovery and evaluation, in which the third step has been altered to now predict efficacy and toxicity in parallel.

FIG. 3 represents hypothetical results from a research protocol in which the CME efficacy is evaluated before toxicity.

FIG. 4 represents hypothetical results from a research protocol in which the CME efficacy is evaluated in parallel with the toxicity.

FIG. 5 presents a flowchart showing an overview of the steps for a portion of a research protocol modified according to the invention.

FIG. 6 presents a flowchart showing an overview of the steps for making a toxicity evaluation according to the invention.

FIG. 7 presents an illustration of the division of a Reference Dataset into a Training Dataset and a Testing Dataset.

FIG. 8 presents a flowchart showing additional detail for the steps for building the toxicity model using machine learning according to the invention.

FIG. 9 illustrates a decision tree for the evaluation of a line of data according to an embodiment of the invention.

FIG. 10 presents a flowchart showing additional detail for the steps for evaluating the data for CMEs and then using toxicity model to predict toxicity according to the invention.

FIG. 11 illustrates an example of a typical toxicity report according to an embodiment of the invention.

FIG. 12 presents a block diagram of the components of a computer system that may be used to execute embodiments of the method of the invention or portions thereof.

### DETAILED DESCRIPTIONS OF EMBODIMENTS OF THE INVENTION

### I. Introduction

The invention disclosed is a method for evaluating candidate molecular entities (CMEs) (generally new chemical compounds, but which may include known compounds as well) for toxicity or other adverse biological effects. The invention makes it possible to rapidly and efficiently evaluate toxicity for a large number of CMEs. Therefore, these methods can be used as part of a drug discovery protocol to determine which potential drug candidates should progress to early-stage animal testing, and which might be eliminated early in the process.

One such research protocol would include evaluation of the compound toxicity alongside compound efficacy, as was suggested in FIGS. 2 and 4. An example of this is illustrated in FIG. 5. In this figure, the initial step 510 is to identify the elements or steps of a research protocol, such as the sequence of trials shown in FIGS. 1 and 2. In the next step 520, the CMEs to be tested are selected. These may be a random assortment of known chemicals, or a library of compounds owned by a pharmaceutical company or developed by a university. In the next step 530, data from the selected CMEs are evaluated for both efficacy and toxicity. In the next step 533, the efficacy and toxicity results are evaluated, and results with various figures of merit are generated. In the last step 535, the CMEs identified as potentially toxic can be removed from the research protocol, saving costs by eliminating the pre-clinical and clinical trials for compounds likely to be toxic.

Some embodiments of the invention would include tests that evaluate a compound's liver toxicity. Other embodiments may include tests that evaluate cardiovascular toxicity, nephrotoxicity, neurotoxicity, or other types of toxicity. Another application for the technology is to use organ toxicity evaluation to determine what chemical compounds are more toxic to tumors, based on the genetic characteristics of the tumors. For example, expression data from biopsy of a tumor could be used to evaluate the effectiveness of different drugs being considered as treatments.

Some embodiments of the invention would evaluate compounds for toxicity only for the purpose of establishing their general value as potential therapeutics, without establishment of their value for any other specific biological function.

The method disclosed here uses relatively large amounts of data with known toxicity results (typically hundreds of thousands of input data rows, each comprising tens of thousands of gene expressions), and applying techniques and algorithms developed for modern machine learning to these datasets to build predictive models.

### II. Overview

FIGS. 6 and 7 illustrate the basic outline of one typical embodiment of the method. An existing Reference Database 1000 of data representing the toxicity results of previously tested compounds is loaded into a model building software program 2000. Examples of such datasets are the Japanese Toxicogenomics Project (JTP) database, which used gene expression data from rat livers and kidneys [T. Uehara et al., “The Japanese toxicogenomics project: application of toxicogenomics”, Mol. Nutr. Food Res. vol. 54, pp 218-27 (2010)] and the xenobiotic and pharmacological liver RNA response dataset created from microarray data by Iconix Biosciences of Foster City, Calif. (now part of Entelos Inc., San Mateo, Calif.) [G. Natsoulis et al., “The liver pharmacological and xenobiotic gene response repertoire”, Mol. Syst. Biol. vol. 4:175, pp. 1-12 (2008)].

Once loaded, as illustrated in FIG. 7, portions of the Reference Dataset 1000 are selected to form a Training Dataset 2111, which is used to train and calibrate various models for toxicity, and other portions (usually the complement of the Training Dataset 2111) are designated for use as a Testing Dataset 2122, to test the models once trained. Typically, the Training Dataset 2111 represents a majority of the data, while the Testing Dataset 2122 is generally smaller.

Returning to FIG. 6, after the training process is completed, a calibrated Toxicity Model 2500 is deployed, now ready to be used on new data from CMEs.

These model building steps may be repeated multiple times, with the Reference Dataset 1000 divided in several different ways and using several algorithms to create the Toxicity Model 2500. This process may be completely automated, but more often, the individual software runs are conducted under human observation, with a trained operator for the software providing input on various ways to divide the Reference Dataset and also select algorithms or models for use.

Meanwhile, CMEs are selected for testing 520, and experiments carried out 526 to generate experimental values for various predictors, such as gene expression results from DNA microarrays. These results then comprise a CME Dataset 1666.

After a step 2600 involving a quality check and consistency control on the CME Dataset 1666, the calibrated CME dataset 2666 is loaded into a computer program 3000, along with the deployed Toxicity Model 2500, and the model used to generate toxicity predictions 3500 for the CMEs represented in the calibrated CME dataset 2666. These results are then analyzed, presented, exported, etc. 3700 to be considered in the evaluation of research protocols.

### III. Generating the Toxicity Model

### III.1. Toxicity Model Reference Dataset

The steps leading to the creation of a Toxicity Model 2500 begin with the identification of a Reference Dataset 1000 for previously tested compounds. Table II shows an example of a few rows of data representative of a typical Reference Dataset 1000. The dataset is structured in rows and columns, with each row typically corresponding to one set of measurements (or “Predictors”) generated for one set of experimental conditions. The illustration of data in Table II represents a simplified illustration; a typical Reference Dataset 1000 may have tens of thousands of rows of data and tens of thousands of columns of data. For a dataset gathered for a rat, there may be 30,000 or more columns of entries. For data gathered for humans, there may 50,000 or more columns of data.

TABLE II
Representative example of eight rows of data as
might be found in a Reference Dataset 1000.

| | Metadata | | | | | | | | Toxicity |
|---|---|---|---|---|---|---|---|---|---|
| ID | | | Sacrifice | | | | | | Results |
| Row | Compound | Dose | Time | | Predictors | | | | Pathology |
| Index | Name | Level | (hr.) | Other | Gene #1 | Gene #2 | Gene #3 | Other | Severity |
| 1 | Aspirin | 0 | 24 | . . . | 0 | 0 | 0 | . . . | 0 |
| 2 | Aspirin | 2000 | 8 | . . . | 2034 | 204 | 2830 | . . . | 0 |
| 3 | Aspirin | 6000 | 8 | . . . | 3523 | 1523 | 930 | . . . | 2 |
| 4 | Vioxx | 0 | 24 | . . . | 0 | 0 | 0 | . . . | 0 |
| 5 | Vioxx | 300 | 8 | . . . | 4393 | 3453 | 8939 | . . . | 2 |
| 6 | Vioxx | 1000 | 16 | . . . | 2039 | 8309 | 8973 | . . . | 4 |
| 7 | Tylenol | 0 | 24 | . . . | 1589 | 1122 | 429 | . . . | 0 |
| 8 | Tylenol | 500 | 24 | . . . | 3108 | 9302 | 1039 | . . . | 2 |

The dataset may have header information that identifies the dataset and provides keys for how various columns may be interpreted. Datasets without header information may also be used.

The leftmost column represents a data row identifier. This could be a single column, or multiple columns, with the data in the column comprising bits representing integers, or floating point numbers, or alphanumeric characters, or other data structures that can uniquely identify a given row.

The columns to the right of the ID information contain identifying information for the experimental data, such as the compound name, along with experimental conditions under which the results were generated. These may also be known as “Metadata”. The Metadata may contain no columns of data describing experimental conditions, or may have many columns describing experimental conditions. These experimental conditions may be the circumstances under which the compound was administered to a test subject, details of the nature of the compound's chemical structure, environmental information for the experiments, or other various measurements that were taken in conjunction with the experiment.

To the right of the Metadata in Table II are columns representing the numerical results for, in this example, various gene expression experiments, represented in this example by integers up to 4 digits long and identified as “Predictors”. These results may DNA microarray and pathology data derived from rat livers or hepatocyte cells (which are derived from the livers of rats by the collagenase perfusion method) or data derived from human hepatocytes. The predictors may be gene expression levels that could be measured by microarray, quantitative polymerase chain reaction (qPCR), high-throughput sequencing or other methods known to those skilled in the art. Gene expression data indicate the extent to which individual genes in an animal or human cell are actively producing RNA.

In some embodiments, the Predictors may represent experimental results related to physiological data taken from a live animal such as cell counts of red blood cells, neutrophils, eosiniphils, basophils, monocytes, lymphocytes, white blood cells, platelets, body weight taken at the time of sacrifice, liver weight, or biochemical tests such as alkaline phophatate, chloride, aspartate or alanine transaminase, gamma-glutaryl transpeptidase, total bilirubin, direct bilirubin, calcium, inorganic phosphate, glucose or creatine kinase concentrations in the blood.

In some embodiments, the Predictors may represent experimental results related to physiological data that represent a characterization of pathological developments in the rat liver which may include (but are not limited to) hypertrophy (swelling of the liver), necrosis (cell death in the liver), microgranuloma (indications of inflammation caused by lymphocytes and macrophages). In some embodiments, the Predictors may represent experimental results that may be an indication of cardiovascular toxicity, nephrotoxicity, or neurotoxicity.

In some embodiments, the Predictors may come from chemical fingerprints of the compound structures. In some embodiments, the Predictors may represent experimental results that include other indications (represented as an integer, decimal number, character string or Boolean) thought to be predictive of compound toxicity. Different measurements may come from different sources and therefore different rows may represent different variables, and so the structure of the database or the various may represent data gathered from more than one source. Although some of the sources of data mentioned above may have come from rats or rat cells, cells from other animals, such as pigs or monkeys, or any other sources believed to be predictive of toxicity may be used.

To the far right of Table II is a column showing data indicating “Toxicity Results”, in this case sub-labeled “Pathology Severity”. Such results may take several different forms, on, perhaps, a scale from 0 (no toxicity) to 100 (lethal), or, as shown, on a scale from 1 to 5, with 1 indicating no toxic effects and 5 being highly toxic. The toxicity results typically have a distinctly different character from that in the other columns, and represent known results from the experimental conditions represented by the Metadata. The Toxicity Results can come from many sources. They can be the observations by a pathologist after viewing a prepared slide from a test subject. They can be the result of mechanical, electrical or chemical instrumentation. They can come from data compiled in reports of drug effects. The Toxicity Results can be qualitative (for example a scale of 0 to 100, 1-4, or an alphanumeric representation (such as +, −, 0; or “very bad”, “bad”, “not bad or good”, “good”, or “very good”). They can include descriptions of the nature of the toxic effect and/or quantitative measurements of toxic effect at the level of molecule, cell, organ, system or organism. There can also be multiple columns of Toxicity Results from multiple experiments. These Results can be provided in a number of different forms. They can be stored electronically in a database along with the Metadata, or in a delimited file associated with the database. They can originally have been derived from paper records.

### III.2. Dividing the Reference Dataset: Metadata and Offset Correction.

The process of training a toxicity model 2500 for deployment is illustrated in more detail in FIG. 8. As discussed above, it begins with the identification of a Reference Dataset 1000. The next step is the importation of the Reference Dataset 1000 into a software program for model building 2000 (which may also comprise a suite of several software programs) running on an electronic computer designed to develop a Toxicity Model 2500. The goal of this software is to calibrate and train a model or set of models. This is achieved by splitting the Reference Dataset 1000 into two subsets—one a Training Dataset 2111 used for training the model, and the other a Testing Dataset 2122, used to test the model once developed, as was illustrated in FIG. 7.

Various preprocessing steps are first carried out after importation of the Reference Dataset 1000. These may include various quality assurance (QA) steps and data scaling and normalization steps. Among these are statistical tests to determine that the data were properly collected, and that the instrumentation was running properly. These tests can include test for consistency between samples and comparisons consistency among measurements made on single samples.

Once the Reference Dataset 1000 has been selected and imported into the software 2000, the next step is a separation 2010 of the Metadata 2011 and Toxicity Results 2012 from the Reference Dataset 1000. A set of decisions needs to be made to determine which variables (e.g. columns of data) of the Reference Dataset 1000 should be considered as Predictors 2041 and which are Metadata 2011. This process may be done automatically, using the recognition of certain patterns of data to identify Metadata 2011, or it may be done with human observation of the data and with real-time editing of the software 2000, or by using an interface with the software designed for that purpose. In the next step 2020, Metadata 2011 (along with toxicity data 2012) can be identified, and analyzed to develop toxicity targets 2088. This listing of the potential target results 2088 can subsequently be used in the training of the models themselves 2400.

For the example shown in Table II, one choice for training targets would be Toxicity Results, such as the column labeled “pathology severity”. All that is required in this case is to pull that single rightmost column of data from the Training Dataset. Another choice might be to build a classifier based on pathology severity greater than or less than 1.5. There are other choices of toxicity targets that may be familiar to those skilled in the art of machine learning, biology or toxicology.

Meanwhile, the next step in the process is a step segregating Control Data 2030 from the rest of the data. Control examples are rows of data representing test subjects (animals or cells that will be for testing new compounds) that have not been given a compound or drug (i.e. zero dose) in order measure those effects that are peculiar to the particular testing and laboratory procedures, or to control for choices for reagents and processing. In the illustration of Table II, each row having a Dose=0 is an example of Control Data 2031.

In the next step, once the control data 2031 has been separated from the remaining Predictor Data 2041, control offsets can be calculated 2035 and then be applied in the next step 2050 to the remaining Predictor Data 2041 to remove any laboratory-specific peculiarities. These offsets could be the average value of the control data 2031, or they could be the median values of the control data 2031, or they could be harmonic means of the control data 2031, or any other calculated method familiar to those who are skilled in the arts of biology and machine learning.

### III.3. Dividing the Reference Dataset: Training and Testing Datasets.

Once a dataset of rows of Predictors has been offset, the next step 2100 is the division of the Predictor Dataset into at least two datasets, a Training Dataset 2111 and a Testing Dataset, as was illustrated FIG. 7.

The choice of which row of data will end up in which Dataset depends on what specific task the predictions be used for. Training objectives might be to predict pathology findings in rat livers, or to predict warning labels assigned by FDA for use in humans. Information considered Metadata, such as Dose Level or Sacrifice time, may or may not be also included as Predictors 2041.

Normally for predictive modeling, each row of the Reference Dataset 1000 would represent an independent measurement. If this is the case, the rows included in the Training Dataset 2111 and those held out for the Testing Dataset 2122 can be chosen at random.

For example, for the example of a Reference Dataset shown in Table II, a possible random division of rows into Training and Testing Datasets might be:

Control Data={rows 1, 4, 7}

Training Dataset={rows 2, 6, 8} (Aspirin, Vioxx, & Tylenol)

Testing Dataset={rows 3, 5} (Aspirin, Vioxx)

(Note: The Control Data may or may not be divided between the Training and Testing Datasets. For the example above, it has been removed prior to random assignment.) The problem with this selection is that the Testing Dataset includes measurements pertaining to Aspirin & Vioxx, which are also compounds represented in the Training Dataset. This means that the Training dataset is not independent from the Testing Dataset, and the Testing Dataset will therefore not provide an independent test of a model trained using the Training Dataset. The model may therefore not give a reliable estimate of toxicity prediction when applied to new compounds.

Therefore, in the current embodiment, when applied to toxicity prediction, the rows from the Reference Dataset 1000 should not necessarily be considered independent from one another. For example in Table II, the first three (3) rows involve Aspirin, the second three (3) involve Vioxx, and the third two (2) involve Tylenol. When building predictive models for predicting toxicity, a separation has to be maintained between the compounds represented in the Training Dataset and Testing Dataset. Beyond the separation of chemical compounds between the two Datasets, the conditions under which these compounds were administered may be different or they may be exactly the same (taken to provide redundant measurements to average out random fluctuations in measurements). Dividing the Datasets by consideration of the experimental conditions may also be desired.

For the example presented in Table II, examples of divisions between Training and Testing Datasets that preserve this separation include:

Training Dataset={rows 1, 2, 3, 4, 5, 6} (Aspirin & Vioxx)

Testing Dataset={rows 7, 8} (Tylenol)

and:

Training Dataset={rows 1, 2, 3, 7, 8} (Aspirin & Tylenol)

Testing Dataset={rows 4, 5, 6} (Vioxx)

and:

Training Dataset={rows 4, 5, 6, 7, 8} (Vioxx & Tylenol)

Testing Dataset={rows 1, 2, 3} (Aspirin)

All of these example divisions exhibit no overlap in the compounds represented in the Training and Testing Datasets. (Note: for these examples, the Control Data has also been divided between the Training and Testing Datasets, with the control data passing to the Dataset associated with the drug study in which the Control Data was gathered.)

There are several ways to break the Reference Dataset 1000 into the Training Dataset 2111 and Testing Dataset 2122 that satisfy the requirement that the Training Dataset 2111 and the Testing Dataset 2122 contain disjoint sets of compounds. One way is to generate a list of all the compounds represented in the Training Dataset. For the example in Table II, that list would be Aspirin, Vioxx, and Tylenol. Then, either automatically within the software or by human input into the computer program, a number or percentage of the compounds that will be represented in the Testing Dataset 2122 can be identified.

The Testing Dataset 2122 is typically smaller than the Training Dataset 2111, comprising, perhaps, 30% of the compounds in the Reference Dataset 1000. That number of compounds may be selected at random from the list of all compounds represented in the Reference Dataset (a random selection of 33% could result in compound B being chosen from the list {A, B, C}, for example). The Testing Dataset 2122 is then assembled by combining all the rows from the Reference Dataset 1000 having those compounds. The remaining rows from the Reference Dataset 1000 are assigned to the Testing Dataset 2111.

This step of data separation 2100 can be conducted a single time. It can also be conducted in a round-robin fashion that is similar to a “cross-validation” method known in the art of machine learning. The round-robin process entails conducting the separation step 2100 described above using a fraction of the compounds from the list (for example, 30% of the compounds), building a model and then testing it, and then repeating the separation 2100 and proceeding to build a second model. Each iteration removes a different subset of rows corresponding to different compounds until all the compounds have been excluded from the Testing Dataset at least once. Each pass through this round-robin process requires retraining (as described below). This increases the computational burden, but has the advantage of providing more thorough statistics on the behavior of the trained models when applied to compounds not in the Training Dataset.

There are several standard variations on this basic process. One variation is to repeat the data separation 2100 and model building process 2200 with multiple variations for the Testing Datasets 2122. With each repetition, the Testing Dataset 2122 is disjoint from the previously selected Testing Datasets. This process is called n-fold cross-validation in the machine learning literature, and it appears to be a very good choice of holdout methods for the problem of predicting drug toxicity. N-fold cross validation takes n passes through the data separation step 2100 through the model building step 2400. In each pass, 1/n th of the Predictors are held out for inclusion in the Testing Dataset. The n sets of Testing Data are disjoint from one another. At the end of n-fold cross-validation, the model complexity that gives the best average performance across the n examples is chosen for deployment as the Toxicity Model 2500. Other variations on this procedure will be known to those skilled in the art of machine learning.

It should be noted that the targets 2088 (generated from the data representing Toxicity results 2012) have been removed at this point from both the Training Dataset 2111 and the Testing Dataset 2122. Values of the targets, however, have not been discarded, but are brought into the procedure and used with the corresponding data during in the model building process.

### III.3 Toxicity Model Building.

The step of building a model 2200 to predict toxicity for new compounds has several stages, as illustrated in FIG. 8. The process incorporates the Training Dataset 2111 and the Testing Dataset 2122 described above.

Initially, the model may have a number of parameters that can be varied, and so can be applied to the Predictors within Training Dataset 2111 to relate them to the target values, in this case Toxicity results set aside as Targets 2088 known to correspond to the rows in the Training Dataset 2111. This may be conceptually thought of as calibrating a huge set of matrix equations, with the model corresponding to a huge matrix operator acting on a matrix of rows of Predictors to result in a corresponding vector of toxicity Targets. With the input Predictors and output Targets known, the model building operation may be thought of as a massive fitting program for the matrix relating them.

A number of different modules using various predictive algorithms can be used as the model. Explication for several options for predictive algorithms is given in Section IV below. In the first step of the model building process 2200, the initial model or algorithm is selected. This can be an automatic selection, based on certain pre-programmed criteria, or it can be selected by human input into the computer program 2000 after an examination of the Reference Dataset 1000 and its various subsets.

A designated algorithm, once chosen, may then be used to build a family of predictive models (not just one). After seeing the results using one algorithm or model, a second algorithm may be selected, again, either by machine or by human operator, and the process repeated to determine if the predictions are more accurate. Using multiple models or algorithms can allow fuller picture of toxicity to be achieved. One model might be trained against classification targets, while another trained against ranking targets. These choices will be familiar to those skilled in the art of machine learning.

FIG. 9 illustrates one example of an algorithm for relating predictors to toxicity results, using a logic tree. The first action is the importation of a row of data 2199 from which has been read 2195 from a dataset 2191. In this case, the illustration is generic—the dataset 2199 may be the Training Dataset 2111, where the values for both Predictors and Toxicity Targets are known, and the coefficients and parameters within the tree (shown in the Fitting Parameters Table within FIG. 9) are adjusted to create the best fit; or the dataset 2199 may be the Testing Dataset 2122, in which only predictors are used to predict toxicity, and the Fitting parameters have been fixed. The results of the logic tree are applied to the various rows of data, and then checked against the known values of toxicity.

Once a row of data from the Dataset 2191 has been imported, the binary decision tree uses a sequence of binary decisions to reach a final prediction. When modeled using the Training Dataset, the input predictors are known, and the resulting toxicity values are known, so the fitting program must find the suitable fitting parameters to create a tree. In the simple illustrative example in FIG. 9, the first binary decision is an evaluation 2230 of Gene #2, namely whether the expression level for Gene #2 is greater than a value B. If that test evaluates to “NO”, then the process proceeds to assign a Pathology Prediction value of “0”. If the results evaluate to “YES”, then the logic tree proceeds to an evaluation 2240 of Gene #1, namely a comparison of the expression level of Gene #1 to a value A. If that comparison comes out “YES”, then the process proceeds to assign a pathology prediction of “2”. However, because there are other outcomes that produce a value of “2”, no definitive statement can be made about A using the limited data shown in Table II. If, however, Gene #1 evaluates to “NO”, then the logic tree proceeds to an evaluation 2250 of Gene #3, namely a comparison of the expression level of Gene #3 to a value C.

Modern machine learning algorithms train multiple models of differing complexity. In this tree, both the sequence of gene tests (e.g. Gene #2 first, then Gene #1, then Gene #3, etc.) and the expression values (in this example, A, B, and C) may be adjusted and fit to best model the data presented in the Training Dataset. The binary tree example in this illustration has a depth of three (3). It is possible to constrain the training of a binary decision tree to consider only trees of depth three (3) or less, or trees of depth four (4) or less etc. In this way a family of models can be generated. The trees that are depth ten (10) are much more complicated than those that are depth three (3). Other possible algorithms or models may generate families of models of differing complexity. The means of generating the model families are familiar to those practiced in the art of machine learning.

Referring again to FIG. 8, in the next step 2400 the model(s), now calibrated using the Training Dataset 2111, are applied to the Testing Dataset 2122. This time, the known Toxicity Targets 2088 are held in reserve, and the results predicted by applying the model to the Testing Predictors within the Testing Data are generated independently of Toxicity Targets 2088. Once calculated, however, the predicted results will be compared with the known targets to determine which model has done the best job predicting toxicity.

There are many performance measures that can be used for this purpose. The performance could be mean square error, mean absolute error, misclassification error, area under the ROC curve, binomial deviance, rank correlation and many other possible choices familiar to those practiced in the art of machine learning.

The properties of the families of models generated by predictive analytics algorithms are described in more detail below. These model families are parameterized by a handful of parameters that are peculiar to the particular algorithm chosen. These parameters can be chosen to yield a model with many degrees of freedom or a model with very few degrees of freedom. The degrees-of-freedom in a model is also called “model complexity”. Which specific model from the family is deployed to make actual predictions on new data is determined by testing all the models against the Testing Dataset. When the Training Dataset has more rows of data, a more complex model with more degrees of freedom will give the best performance on the Testing Dataset. When the Training Dataset has fewer rows of data, then less complex model with fewer degrees of freedom will give the best performance on the Testing Dataset.

As depicted in FIG. 8, the final step 2450 in arriving at a model for predicting toxicity is to use the Testing Dataset 2122 to pick the best model from the models tested. The output of this selection is the Toxicity Model 2500 which will be subsequently used to make toxicity predictions for data related to new compounds.

### IV. Algorithms

The approach presented here to evaluate toxicity uses techniques developed in the field of machine learning. There are many algorithms and models that have been developed for machine learning to solve other problems, and the embodiments of the invention presented here can use any or all of these algorithms and models, and benefit from the experience they offer. [For a more general reference on machine learning, see Chapters 3 and 4 of The Elements of Statistical Learning: Data Mining, Inference, and Prediction (2^{nd} Ed) by T. Hastie, R. Tibshirani, and J. H. Friedman (Springer Verlag, Springer Series in Statistics, (2008)). For a reference on specific algorithms, see R. Caruna, N. Karampatziakis & A. Yessenalina, “An Empirical Evaluation of Supervised Learning in Higher Dimensions”, in the Proceedings of the 25th International Conference on Machine Learning. (ACM, New York, N.Y., 2008)].

Predictive analytics models, such as those used in the embodiments of the invention described in this application, comprise a collection of parameter values. The number and meaning of the parameters will vary from one predictive analytics algorithm to another.

### IV.1 Regression Background.

“Regularized Regression” describes a class of methods for adding a complexity control parameter to ordinary least squares regression. The ordinary least squares regression problem is described as follows:

Suppose the data consist of n input data instances {xᵢ, yᵢ} for i=1 to n. Each instance includes a vector of p predictors (or regressors) xᵢ and a scalar measure of toxicity yᵢ that will be used to predict toxicity for new compounds. Referring to example data presented in Table II, the vector of predictors are the entries in the ith row under the headings “Gene#1 Expressions Level, Gene#2 Expression Level . . . ” and the scalar measure of toxicity yᵢ is the number in the ith row under the heading “Pathology Severity”.

In a linear regression model the toxicity is a linear function of the regressors:

yᵢ=xᵢ′β+εᵢ

where β is a p×1 vector of unknown parameters; the εᵢ terms are unobserved scalar random variables (errors) that account for the discrepancy between the actually observed toxicities yᵢ and the “predicted toxicities” x′₁β; and ′ denotes matrix transpose, so that x′β is the dot product between the vectors x and β. This model can also be written in matrix notation as:

y=Xβ+ε

where X is a matrix whose rows are the vectors xᵢ from the equation above. Again referring to Table II, the vector y is the vector of entries under the column heading “Pathology Severity” and the matrix of predictors X is the matrix of numbers under the column headings “Gene#1 Expression Level, Gene#2 Expression Level, . . . ”.

Ordinary least squares regression selects the elements of the coefficient vector β by solving the problem of minimizing the sum of squared errors (i.e. sum of εᵢ²). In the problem of predicting toxicity, given the data available ordinary least squares can fit the training data too well, and yield performance on new drug data that is wildly in error. For this case, ordinary least squares yields a model that is too complex.

Regularization introduces additional constraints on the ordinary least squares problem in order to get control of model complexity in ordinary least squares regression.

### IV.2. Best Subset Selection Algorithm.

One method called “best subset selection” works as follows. To the ordinary least squares problem, add the additional constraint that all but one of the elements of the coefficient vector β are 0. Only one of the items from the input data vector xᵢ will be used to estimate toxicity. All of the others will be ignored. With that constraint it can be determined which input is the best one, and what value the corresponding element of the coefficient vector β should have. We could also make the constraint that all but two elements of the coefficient vector β are 0. Continuing in this way gives us a family of different models. We can use performance on out-of-sample data to determine how many non-zero elements of β gives the best performance on data for new compounds.

In the problem of predicting toxicity, the number of elements in a single Reference Dataset (the dimensionality of the regressor) can be several tens of thousands. The large numbers of potentially useful input variables can come from a variety of sources (e.g. gene expression data or molecular signatures).

### IV.3. Coefficient Penalized Linear Regression Algorithms.

IV.3.a. Lasso Regression Algorithm.

Coefficient penalties are another way to introduce a complexity parameter into linear regression. There are a variety of different Coefficient Penalized Linear Regression algorithms. In contrast to subset selection, which forces coefficients to be either “on” or “off”, coefficient penalized linear regression places a penalty on the aggregate value of the coefficients. It is acceptable for all coefficients to be “on” as long as they are all small.

This works by solving a constrained minimization problem. Minimize the ordinary least squares criterion subject to a constraint on the aggregate magnitude of the coefficients. In mathematical terms the problem is as follows. Build a model of toxicity as a linear function of the input data:

y=Xβ+ε

and then minimize the sum of squared errors (i.e. sum of εᵢ²) subject to the constraint that the norm of the coefficient vector satisfies:

∥β∥₁=λ

Now the parameter λ becomes the complexity parameter. The norm bars ∥ ∥ are drawn with a subscript “1” to indicate the l₁ norm; that is, the sum of the absolute values of the components of the coefficient vector β. Using the l₁ norm for the coefficient constraint results in a method also known as “Lasso Regression”. This method has the property that resulting coefficient vectors can be sparse.
IV.3.b. Ridge Regression Algorithm.

Another choice of coefficient penalty is the l₂ norm squared—that is the sum of the squared coefficients. In that case, the constraint equation becomes:

∥β∥₂²=λ.

The algorithm using this form of penalty goes by the name of “Ridge Regression”.
IV.3.c. ElasticNet Regression Algorithm.

Yet another choice of coefficient penalty is a blend of l₁ and l₂ penalties. In this case an additional parameter α is introduced where 0<α<1. The coefficient penalty is then given by:

(1−α)∥β∥₂²+α∥β∥₁=λ

The choice of α determines how whether how the lᵢ and l₂ penalties are blended for the final penalty. This makes it possible to alter the character of solutions in order to achieve best performance. The algorithm using this form of penalty is called “ElasticNet”.
IV.3.d. Application of Coefficient Penalized Linear Regression Algorithms.

Whichever penalty method is chosen, the procedure for developing a trained model is as outlined above. The data for the compounds of the Reference Dataset are divided into a Training Dataset and Testing Dataset. All of the rows in the Reference Dataset are assigned to one of these two datasets, so that there are no compounds in common between the Training Dataset and the Testing Dataset, as described above. Then, the coefficient penalized linear regression problem is solved for a variety of different constraint values (called λ for the coefficient penalized linear regression methods). That is, solve the constrained minimization problem for a variety of constraint values. (Notice that for each algorithm, the nature of the constraint is different.) This gives a family of models parameterized by the constraint value. Then, these models are applied to the Testing Dataset, and the ability of the various models to predict the known target for toxicity is evaluated. From these results, one of these models is selected—that is, select the constraint value λ, for which the corresponding model yields lowest error on the out of results from the testing Dataset. The selected model is the one that is most likely to give the best performance when applied to data from new compounds. Once the model is selected, then it can be used to make predictions.

Training the model, and applying the model to the Testing Dataset, can be done on a different computers or the same computer. Likewise, the application of the deployed Toxicity Model to any dataset of new compounds may be carried out on the same computer as was used for training and testing, or using a different computer. The training and selection process is more CPU intensive than making predictions with the model, so although a single CPU may be used, a CPU with multiple cores may also be used, and multiple CPUS or even multiple computer systems operating in parallel may be used to perform these calculations. Depending on the size of the dataset, model training can be done on a CPU or set of CPUs running any operating system that supports a compiler or interpreter for R, Python®, C, C++, Java®, JavaScript®, or any other programming language suitable for mathematical programming. Among these operating systems are Linux®, UNIX®, Oracle Solaris and its variants, Windows® and its variants, Mac OS-X®, etc. The training can also be done on multiple CPUs, as some of the algorithms for solving the constrained minimization problem can easily be fit into a map-reduce paradigm and programmed for multiple CPUs. Either training or prediction can also be done remotely or using remote computing or memory resources “in the cloud”.

### IV.4. The Glmnet Algorithm

The glmnet algorithm is an algorithm for solving the constrained minimization problem for Lasso, Ridge and ElasticNet regression. It is a particularly fast algorithm that facilitates the rapid production of trained models on Reference Datasets that have many rows and many columns. [For more on the glmnet algorithm, see J. Friedman, T. Hastie & R. Tibshirani, “Regularization Paths for Generalized Linear Models via Coordinate Descent”, J. Stat. Softw. vol. 33 pp. 1-22 (2010)].

### IV 5. Algorithms Based on Binary Decision Trees.

IV.5.a. Binary Decision Tree.

Another class of predictive machine learning algorithms that can be used to predict toxicity is binary decision trees and ensembles of binary decision trees. These algorithms start with the same datasets as before. Once a binary decision tree has been trained it has a form such as the one illustrated in FIG. 9.

The trained tree is used to make predictions based on the variables available. These include both conditions and measurements that are present in the Dataset 2191 indicated in FIG. 9. As explained above, these variables are present in the Reference Dataset, the Training Dataset and the Testing Dataset, and must match format, data type, scaling etc., as is also described above. The inputs to the trained binary decision tree may also be rows from the CME Dataset 1666, as will be described below.

At each level of the tree, a Boolean statement is posed regarding one of the variables in the CME Dataset by comparing the variable to the possible values that the variable can take. For numeric variables the comparison is of the form
variable>value.
   (the relationship can also be “greater than or equal to”, “less than”, “less than or equal to” etc.). If the variable takes discrete values that are unordered (like TRUE/FALSE, or “Male”, “Female” or “Hermaphrodite”) then the comparison is set theoretic inclusion in a subset of possible values that the variable can take (e.g. if the possibilities for the variable are “Male”, “Female” and “Hermaphrodite”, then the comparison might be variable a member of {“Male”, “Hermaphrodite” }). The Boolean value that statement takes for the particular row being considered determines whether the left path or right path down the tree is taken, as indicated in FIG. 9. Taking the appropriate path leads to one of two things—either another binary statement about one of the variables from the row being considered or the path terminates at what is called a “leaf node”. Each leaf node associates a predicted value to all the rows that wind up at that leaf.

Training a binary decision tree is a recursive process that uses the Training Dataset, which includes, as explained above, the known toxicity outcome values for all the rows. To determine the first variable and the value against which it will be tested, the training process enumerates all the possible choices of variable. If the variable admits an ordering (i.e. if “<” is meaningful, as it is for integers or real numbers) then all meaningful possibilities x for the statement “variable<x” are attempted. If the variable doesn't admit an ordering (as for “Male”, “Female”, “Hermaphrodite”) then all possible subsets of the variable values are attempted. For each variable all the attempted binary decisions on that variable the performance of the resulting split is tested against all the rows in the Training Dataset. This process results in the rows of Training Dataset being split into two subsets for each choice of variable and Boolean test. The performance of each split is measured against these two subsets. That performance is measured in one of two ways depending on the nature of the toxicity outcome.

Assessing the performance of the splits uses the toxicity outcomes that are available for the Reference Dataset and therefore for the Training Dataset and the Testing Dataset. If the toxicity outcomes are two-valued (toxic?=Yes or No), then the purity of the subsets resulting from the split is used to determine how well the split work. Frequently used measures of purity include entropy, misclassification error and the so-called GINI index. If the toxicity values are real-valued (e.g. a toxicity level from 0 to 4), then the sum-squared error of the splits can be used to determine split performance. This exhaustive process determines the best choice for the first variable and the Boolean test that will be applied to that variable to split. The same process is then applied to each of the two subsets that result from the first split. The process continues in this recursive manner until stopping conditions are met.

The binary tree can be stopped using several criteria. It can be stopped at a fixed depth. Stopping at depth two (2) would result in a maximum of two decisions along any path down the tree. Tree building can also be stopped when the number of instanced resulting from a split are too few. It can also be stopped if the improvement in purity or sum-squared error becomes too small. All of these parameters can work as complexity parameters for a binary decision tree. The Testing Dataset that was held out at the beginning can be used to determine the best choices for these complexity parameters for growing the tree that will actually be used to make predictions on data from new drugs.

IV.5.b. Ensemble Methods.

In addition to single binary trees as described above, binary trees are also used in what are called “ensemble methods”. Since ensemble methods incorporate multiple binary trees, they inherit the many of the properties of binary trees. The idea for ensembles stems from results in computational learning theory. These results establish that using a large number of models, none of which is spectacular, but which are independent of one another, can result in very good performance. This has led to a number of very powerful predictive algorithms based on growing large numbers of binary trees. The trick with these ensemble methods is to figure out a way to systematically generate a large number of trees that are all trying to solve the same problem, but which are not identical to one another. There are several established ensemble methods. A single ensemble method may be used, or various methods may be used in combination. Some basic ensemble methods are called “bagging”, “random forests”, “gradient boosting” and “stochastic gradient boosting”.

IV.5.b.i. Bagging

With “Bagging”, the idea is to build hundreds or even thousands of trees, all built on different random samples of the Training Dataset. So the process is as follows for bagging: take a random sample of the rows from the Training Dataset, and use the recursive process to build a binary decision tree. Take another random sample from the Training Dataset, and build a second binary decision tree. Continue this process until several hundred or even thousands of trees have been built. Then, the average of the predictions from these individual trees is calculated to produce a final prediction.

IV.5.b.ii. Random Forests

“Random Forests” uses a different method to generate independent trees. At each stage of growing the individual trees, Random Forests removes some of the input variables from consideration as the split-point variable. This has the benefit of making the trees faster to grow and may be part of the reason that Random Forests has been successful on very high-dimensioned input data and on input data that is sparse (mostly zeros). Since the selection of ignored variables is random, all of the variables are represented in many of the trees, just not in all of them. Random Forests can also include the Bagging process of additionally selecting a random portion of the Training Dataset. Once the trees are grown the Random Forests procedure is to average the tree outputs in order to form a final prediction.

IV.5.b.iii. Gradient Boosting and Stochastic Gradient Boosting

“Gradient Boosting” methods, also known as “gbm”, build an ensemble of trees by iteratively building each tree to predict the accumulated errors of all the earlier trees. Let Tᵢ be the ith tree built by the gradient boosting process, and let Tᵢ(X) be the toxicity predictions generated by the ith tree when applied to the Training Dataset. As an example of the function T( ), the tree depicted in FIG. 9 maps predictor gene expression values to values for “Pathology Prediction”. Then the first tree is built using the basic recursive tree-building algorithm on the Training Dataset and the known toxicity results (called X and y above). The second tree T₂ uses the same matrix of predictors X, but is built to predict the errors left over from the first tree y−εT₁(X). The parameter ε is required to insure convergence of the sequence of approximations and is usually best set somewhere between 0.001<s<0.1. At the nth step in the iteration can be written as follows:

Initialization:
Build first tree T₁ to predict y from X

Let P₁=εT₁(X)

Iteration:
Build Tₙ to predict y−Pₙ₋₁ from X

Let Pₙ=Pₙ₋₁+εTₙ(X)

Stochastic Gradient Boosting introduces the additional step of taking a random subset of the Training Dataset before building each of the Tₙ. [For more on gradient boosting methods, see J. H. Friedman, “Greedy Function Approximation: A Gradient Boosting Machine”, Annals of Statistics, vol. 29, pp. 1189-1232 (2000); and “Stochastic Gradient Boosting”, Comput. Stat. Data Anal. vol. 38, pp. 367-78 (1999)].

### IV.6. Other Predictive Algorithms.

Several other predictive modeling algorithms may be used for making toxicity predictions, such as the “Support Vector Machine”. Another algorithm is called a “Neural Net”, which has a variety of types, including feed-forward neural nets, recurrent neural nets, restricted Boltzmann machines, deep belief networks and auto encoders. Other possible algorithms will be known to those skilled in the art of machine learning.

### IV.7. Combinations of Algorithms

Different algorithms have different strengths. Sometimes combinations of them can lead to improved performance. Coefficient penalized linear regression algorithms can be solved very rapidly using the glmnet algorithm, but the resulting models are linear and do not account for interactions between variables. These interactions can be accounted for by what is called “basis expansion” (expanding the number of columns in the Training Dataset by including all pair-wise products of measurements and conditions). In some cases “basis expansion” leads to billions of columns in the resulting dataset and becomes unwieldy.

Another approach would be to use one of the tree-based ensemble methods, which more naturally include variable-variable interactions, but those methods can be slower to train on large datasets. Another approach is to use one of the coefficient penalized linear regression algorithms to determine which columns from the Reference Dataset are most important and then to use that subset of columns in training one of the tree-based ensemble methods, which will incorporate the variable-variable interactions.

Modern predictive algorithms have the property that they are built and judged on statistical measures of how well they predict outcomes. These models do not depend on prejudging which input variables will be important.

### V. Applying the Model to New Data

### V.1. Creating a New Compound Dataset.

Once the toxicity model 2500 has been calibrated and deployed, it can be applied using a similar software program to the CME Dataset 1666 to make toxicity predictions for new compounds. Referring again to FIG. 6, in the initial step 520 of this activity, new molecular entities (CMEs) or chemical compounds are selected for toxicity testing. The new compounds maybe created by drug chemists based on similar compounds already in use. They may be chemicals harvested from natural sources. They may be molecules constructed for a particular molecular shape, in order to bind with a specific target related to a disease pathway. Or, they may be derived from other sources familiar to those skilled in the arts of botany, chemistry, or drug discovery.

In the second step 526, lab processes are carried out using the selected new compounds in order to extract quantitative and qualitative data to be used for predicting toxicity. It may include dosing animal or human cells with the CMEs. These cells may be from specific organs, such as livers, kidneys, hearts, or brains. The cells may be specific target cells such as hepatocytes or groups of cells that interact in ways that are suspected to be toxic by those skilled in the art.

The lab processes may also include dosing live animals with the CMEs. The quantitative and qualitative data may include physiological measurement such as change in body weight, blood chemistry and other physiological measurements familiar to those skilled in the art. The quantitative and qualitative data may include data from a trained pathologist's evaluation of specific organs such as liver, heart, kidney, or brain. The quantitative and qualitative data may include a pathologist's evaluation of specific cells. It may also include automated evaluation by means of image processing of images from target organs, cells or groups of cells, such as gene expression data gathered using DNA microarrays and the like. The quantitative and qualitative data may include descriptions of compound molecular structure or the atoms making up the molecule or both structure and composition.

In some embodiments of the invention, microarray data derived from rat or human hepatocytes exposed to the chemical compound in culture medium at a single concentration or range of concentrations (which include physiological or biochemical activity) may be used. Another embodiment of the invention may use microarray data derived from the liver organ itself after the host animal had been exposed to the chemical compound. Another embodiment of the invention may use chemical fingerprint data derived from the chemical compound to the model to determine its toxic properties. Another embodiment of the invention may use physiological data taken from an animal that had been exposed to the chemical compound.

The result of these experimental measurements is a CME Dataset 1666. Table III shows an example of a few rows of data representative of a typical CME Dataset 1666. The dataset is structured in rows and columns, with each row typically corresponding to one set of measurements (or “Predictors”) generated for one set of experimental conditions (listed as “Metadata”). In the example shown in Table III, the metadata may include things like the names (or some other identification) of the new compounds being considered. It may include other items such as dose level or the time that the animal or cell culture was subjected to exposure to the New Compound. Rows of Control Data, with dose levels set to zero, may also be included.

TABLE III
Representative example of eight rows of data
as might be found in a CME Dataset 1666.

| | Metadata | | | | | | | |
|---|---|---|---|---|---|---|---|---|
| ID | | | Sacrifice | | | | | |
| Row | Compound | Dose | Time | | Predictors | | | |
| Index | Name | Level | (hr.) | Other | Gene #1 | Gene #2 | Gene #3 | Other |
| 1 | AAA | 0 | 24 | . . . | 0 | 0 | 0 | . . . |
| 2 | AAA | 3000 | 8 | . . . | 2342 | 902 | 98900 | . . . |
| 3 | AAA | 10000 | 24 | . . . | 523 | 79802 | 890 | . . . |
| 4 | BBB | 0 | 24 | . . . | 0 | 0 | 0 | . . . |
| 5 | BBB | 1000 | 8 | . . . | 10983 | 1903 | 2893 | . . . |
| 6 | BBB | 3000 | 16 | . . . | 1832 | 29030 | 7090 | . . . |
| 7 | CCC | 0 | 24 | . . . | 3619 | 44 | 193 | . . . |
| 8 | CCC | 3000 | 24 | . . . | 2992 | 29 | 302 | . . . |

The predictors may be gene expression levels that could be measured by microarray, quantitative polymerase chain reaction (qPCR), high-throughput sequencing or other methods known to those skilled in the art. Gene expression data indicate the extent to which individual genes in an animal or human cell are actively producing RNA.

Other predictors of toxic outcomes might be used instead of or in addition to gene expression data. These might include chemical structure data for the New Compounds, measures of blood chemistry for animal or human test subjects, measures of animal, organ or cell pathology that are different from the Toxicity Outcome being predicted by the model or other available predictors known to practitioners of the art. The simple illustrative example in Table III shows eight (8) rows of data with data for expression levels for only three (3) genes. The illustration of data in Table III represents a simplified illustration; a typical CME Dataset 1666 may have tens of thousands of rows of data and tens of thousands of columns of data. For a dataset gathered for a rat, there may be 30,000 or more columns of entries. For data gathered for humans, there may 50,000 or more columns of data.

The structure of the CME Dataset 1666 is essentially the same as that used to represent the Reference Dataset 1000, as was illustrated in Table II, with columns representing ID, Metadata and Predictors. However, for these new compounds, toxicity is unknown, and therefore there is no column representing toxicity data.

Instead, the Predictor data indicated in Table III are those data used by the deployed Toxicity Model 2500 to make a prediction of toxicity outcome for the New Compound. The deployed Toxicity Model 2500 uses these data to make predictions of toxic outcomes for new compounds.

This CME Dataset 1666 can be stored on digital media such as hard drive, removable hard drive, USB thumb drive or RAM or other digital media used by those skilled in the art. They may be stored in the form of a delimited file or database. Although data structures using rows and columns have been shown, the data may be arranged in different schemata that will be known to those skilled in the art.

Regardless of the schema used, it is important that the CME Dataset 1666 have the same format as the Reference Dataset 1000 in several regards. It is preferred that the CME Dataset 1666 have the same data types in the same places as the Reference Dataset 1000. Likewise, it is preferred that the numeric values be scaled in the same way for the two datasets. If certain algorithms have been deployed, the names of the variables must be present and match in order insure that columns of data correspond appropriately. It is therefore typical that, in most embodiments of the invention, the CME Dataset 1666 is checked 2600 for conformance with the structure of the Reference Dataset 1000.

This checking process 2600 is shown in more detail in FIG. 10. Once the CME Dataset 1666 has been imported into the software doing the quality checks, one or more quality assurance (QA) steps 2610 are carried out. Among these are statistical tests to determine that the data were properly collected, and that the instrumentation was running properly. These tests can include test for consistency between samples, and comparisons for consistency among measurements made on single samples.

The program will evaluate if there are any fundamental inconsistencies within the data and its formatting, and determine if the data are acceptable in the next step 2620. In some instances, if some rows deviate too far from statistically expected behavior, they may be discarded 2625, and the revised Dataset re-submitted to QA testing. If the data are then acceptable, the program will then proceed to the next steps of the data checking and refinement process 2600, in which offsets are refined and adjusted.

However, if there is a problem with the data that cannot be normalized or corrected with the pre-programmed procedures, the program will stop 999 and the human operator alerted. The operator may respond by editing or reformatting the data within the CME Dataset 1666, and then running the QA steps 2610 again.

Continuing in FIG. 10, the next step 2630 separates the control data 2631, which is used in the next step 2635 that generates offset variations from one laboratory test run to another. This process is analogous to the offset correction 2035 carried out on the Reference Dataset 1000, and in some embodiments will use the exact same code using the exact same offset procedures. The offset result is then applied to the predictors 2641 that were left after the Control Data 2631 was removed, resulting in calibrated Predictors 2666.

### V.2. Predicting Toxicity.

Continuing with FIG. 10, once the calibrated Predictors 2666 have been formed from the CME Dataset 1666 by offset and correction, the Toxicity Model 2500 can now be applied to predict toxicity results from the Predictor values. A computer program 3000 stored on digital medium either on a computer storage system, or in a remote storage location accessible through the Internet or another network, apply the Toxicity Model 2500 comprising algorithms previously calibrated using the Training Dataset 2111 and the Testing Dataset 2122, and calculates a prediction of the Toxicity Outcome using the calibrated Predictors 2666 as the input. The output of the toxicity prediction 3300 can be predictions of the toxicity outcome or it can be probabilities for several possible toxicity outcomes.

As one specific example of this process, refer again to FIG. 9, which illustrates how the process works for a single binary decision tree using gene expression data to predict pathology as a Toxicity Outcome. The deployed Toxicity Model 2500 in this example consists of applying a series of binary decisions using specific, calibrated inequality values to each line of the Dataset 2191. In this case, however, the Dataset 2191 used as input as the set of calibrated predictors 2666.

Table IV illustrates a representation of a dataset of calibrated Predictors 2666 derived from the CME Dataset illustrated in Table III, along with the corresponding predicted toxicity results. For this table, the entries for the Control Data have either remained 0, or (as is the case for the data on compound CCC), the values have been normalized by subtracting the Control Data values for this compound as listed in Table II.

TABLE V
Representative example of 8 rows of data from after the prediction
of toxicity results. To the left are the columns containing ID data
and the Calibrated Predictors 2666, and to the right the raw toxicity
predictions 3500 according to the Tree shown in FIG. 9.

| | | | | | | Toxicity |
|---|---|---|---|---|---|---|
| ID | Metadata | | | | | Results |
| Row | Compound | Calibrated Predictors | | | | Pathology |
| Index | Name | Gene #1 | Gene #2 | Gene #3 | Other | Severity |
| 1 | AAA | 0 | 0 | 0 | . . . | 0 |
| 2 | AAA | 2342 | 902 | 98900 | . . . | 0 |
| 3 | AAA | 523 | 79802 | 890 | . . . | 2 |
| 4 | BBB | 0 | 0 | 0 | . . . | 0 |
| 5 | BBB | 10983 | 1903 | 2893 | . . . | 2 |
| 6 | BBB | 1832 | 29030 | 7090 | . . . | 4 |
| 7 | CCC | 0 | 0 | 0 | . . . | 0 |
| 8 | CCC | −627 | −15 | 109 | . . . | 0 |

The calculated toxicity results to the right of the Table are generated using the Toxicity Decision Tree in FIG. 9. As a result, Rows 2 and 8 enter Gene #2 test 2230 and result in a NO, and assign a pathology prediction=0, while Rows 3, 5 and 6 would pass the Gene #2 test with a YES, and proceed to the second decision 2240. This second decision 2240 relates to Gene #1, and only Row 5 passes the test with a YES, resulting in a pathology predictor=2, while Rows 3 and 6 result in a NO result for Gene #1, proceeding to the Gene #3 test 2250. This third decision 2250 relates to Gene #3, and only Row 6 results in a YES, resulting in a pathology predictor=4, while Row 3 results in a NO result for Gene #3, resulting in a pathology predictor=2.

The deployed Toxicity Model 2500 from FIG. 9 used in Table IV is a single binary decision tree. Usually better performance is achieved using a multitude of binary decision trees and combining their predictions to yield a composite prediction. Methods for training and combining multiple binary decision trees are called ensemble methods. Several of these methods have been disclosed in Section IV above.

Referring again to FIG. 10, the toxicity predictions 3500 generated for each row in the Predictor Dataset 2666 are aggregated, analyzed and post-processed to produce toxicity summaries 3700 and reports 3800. As examples, CMEs may be compared for toxicity with one another, or ranked relative to one another. The predicted toxicities may be plotted versus dose levels to produce dose response curves for toxic effects. New Compounds may be compared to previously tested compounds.

An example of a graph as might typically be presented in a Toxicity Report is shown in FIG. 11. Here, the prediction of toxicity for various doses of acetaminophen is shown for various values of metadata variables. Reports may have one graph or multiple graphs, one table or multiple tables, and various presentations of printed results, as will be known to those skilled in the art.

Visualization methods 3900 may be applied to the raw data in order to produce summaries, graphs, interactive HTML files and other means of displaying, summarizing and displaying the data. The raw toxicity prediction data may be aggregated by compound and plotted as a function of dose in order to produce a toxicity dose response curve. The characteristics of the trained toxicity model 2500 may be extracted to determine what predictor values are most influential in each of the toxicity predictions or to relate toxicity predictions on new compounds to toxicity predictions on new compounds.

Once determined by the model, the estimate of the toxicity of the compound given by the model is rendered into a result predicting the severity of a toxic response of liver, kidney, heart, neural tissue, or other organ or tissue. The result may take the form of a probability of overall toxicity from a model for a set of possible toxic responses. The result may be embodied in a list of more specific pathologies such as hypertrophy, cellular necrosis, microgranuloma, cellular change, degeneration and other diagnostic terms describing tissue degeneration or pathology in use by medical pathologists. The result may be embodied as a plot of probability of a toxic pathological response plotted over different dosages or time points over the course of an experiment exposing a chemical compound to cells in a culture or a whole animal or control experiments where no compound is present for the sake of comparison to compound effects on cells in a culture or a whole animal. In a computer-implemented embodiment of the invention, the result may be communicated to a database, to a web page or to a text file. The result may be rendered in terms of toxic response at the level of specific cells or groups of cells, or at the level of specific organs or groups of organs. The result may also be rendered at the level of an entire organism (e.g. rat dog, monkey, human, etc.). The result may also be rendered in terms of toxicity comparisons between CMEs. For example, it may present data representing that CME 1 at dose X leads to more liver toxicity than CME 2 at dose Y. Again, these comparisons can be presented in terms of toxicity indications in the cell, organ, or whole animal.

Additionally, the toxicity results may be summarized by the presentation of model behaviors that determine characteristic patterns of groups or sub-groups of genes, whose expression levels, when viewed together, indicate “syndromes” that may indicate toxicity. For example, if Gene #29, Gene #502, and Gene #888 all markedly track each other when the toxic effects are high, these can be labeled as a syndrome to mark toxicity, even if any one of these genes does not in and of itself raise any toxicity warnings.

### VI. Using the Results

The exported result report 3800 may be used in combination with assays of biological or pharmacological activity that evaluate the pharmacological efficacy of the compound in the context of that same compound's toxic effects on humans or animal models. Then the result exported could be used in the context of a research evaluation protocol, as was shown in FIGS. 2 and 5, prior to the compound being prepared to be evaluated by a live animal study. The result exported may be used in conjunction with a ‘hits to lead’ study as illustrated in FIGS. 1 and 2, where new variants of a chemical compound's molecular structure are produced to optimize efficacy, activity or toxicity to a desired level. In some embodiments, the combined decision-making protocol may use toxicity results exported for consideration in combination with a different efficacy or activity. In some embodiments, the chemical compound's toxicity properties may be evaluated without evaluation of pharmacological efficacy or biological for the purpose of understanding the value of the compound's potential for safe human use or ingestion.

### VII. Embodiments on Computers

Although the embodiments disclosed so far comprise the use of a computer for making toxicity predictions, many computers, as well as computers connected through a network such as the Internet, may also be used for to calculate the same or similar results.

FIG. 12 illustrates a block diagram of an exemplary computer system that can serve as a platform for portions of embodiments of the present invention. Computer code in programming languages such as, but not limited to, R, Python®, C, C++, C#, Java®, JavaScript®, Objective C®, Perl®, Boo, Lua, Basic, assembly, Fortran, APL, etc., and executed in operating environments such as UNIX®, Linux®, Oracle Solaris and its variants, Windows® and its variants, Mac OS-X®, as well as iOS®, Android®, Blackberry®, etc., can be written and compiled into a set of computer or machine readable instructions that, when executed by a suitable computer or other microprocessor based machine, can cause the system to execute the methods of the disclosed invention, or subsets thereof.

One embodiment of such a computer system 7000 comprises a bus 7007 which interconnects major subsystems of computer system 7000, which typically comprises: a central processing unit (CPU) 7001; a system memory 7005 (typically random-access memory (RAM), but which may also include read-only memory (ROM), flash RAM, or the like); an input/output (I/O) controller 7020; one or more data storage systems 7050, 7051 such as an internal hard disk drive or an internal flash drive or the like; a network interface 7700 to an external network 7770, such as the Internet, a fiber channel network, or the like; and one or more drives 7060, 7061 operative to receive computer-readable media (CRM) such as an optical disk 7062, compact-disc read-only memory (CD-ROM), compact discs (CDs), floppy disks, universal serial bus (USB) thumbdrives 7063, magnetic tapes, etc.

The computer system 7000 may also comprise: a keyboard 7090; a mouse 7092; and one or more various other I/O devices such as a trackball, an input tablet, a touchscreen device, an audio microphone and the like. These I/O devices may be internal to the system, as is found, for example, if the computer system 7000 is a laptop, or may be external to the system, as is found in typical desktop configurations. The computer system 7000 may also comprise a display device 7080, such as a cathode-ray tube (CRT) screen, a flat panel display or other display device; and an audio output device 7082, such as a speaker system. The computer system 7000 may also comprise an interface 7088 to an external display 7780, which may have additional means for audio, video, or other graphical display capabilities for remote viewing or analysis of results at an additional location.

Bus 7007 allows data communication between central processor 7000 and system memory 7005, which may comprise read-only memory (ROM) or flash memory, as well as random access memory (RAM), as previously noted. The RAM is generally the main memory into which the operating system and application programs are loaded. The ROM or flash memory can contain, among other code, the basic input/output system (BIOS) that controls basic hardware operation such as the interaction with peripheral components. Applications resident within computer system 7000 are generally stored on storage units 7050, 7051 comprising computer readable media (CRM) such as a hard disk drive (e.g., fixed disk) or flash drives.

Data can be imported into the computer system 7000 or exported from the computer system 7000 via drives that accommodate the insertion of portable computer readable media, such as an optical disk 7062, a USB thumbdrive 7063, and the like. Additionally, applications and data can be in the form of electronic signals modulated in accordance with the application and data communication technology when accessed from a network 7770 via network interface 7700. The network interface 7700 may provide a direct connection to a remote server via a direct network link to the Internet via an Internet PoP (Point of Presence). The network interface 7700 may also provide such a connection using wireless techniques, including a digital cellular telephone connection, a digital satellite data connection or the like.

Many other devices or subsystems (not shown) may be connected in a similar manner (e.g., document scanners, digital cameras, etc.). Conversely, all of the devices shown in FIG. 12 need not be present to practice the present disclosure. In some embodiments, the devices and subsystems can be interconnected in different ways from that illustrated in FIG. 12.

Code representing software instructions to implement embodiments of the present invention can be stored on one or more computer-readable storage media such as: the system memory 7005, internal storage units 7050 and 7051, an optical disk 7062, a USB thumbdrive 7063, one or more floppy disks, and the like. The operating system provided for computer system 7000 may be any one of a number of operating systems, such as UNIX®, Linux®, Oracle Solaris, MS-DOS®, MS-WINDOWS®, OS-X® or another known operating system.

Moreover, regarding the signals described herein, those skilled in the art will recognize that a signal can be directly transmitted from one block to another, between single blocks or multiple blocks, or can be modified (e.g., amplified, attenuated, delayed, latched, buffered, inverted, filtered, or otherwise modified) by one or more of the blocks. Furthermore, the computer as described above may be constructed as any one of, or combination of, computer architectures, such as a tower, a desktop, a laptop, a workstation, or a mainframe (server) computer. The computer system may also be any one of a number of other portable computers or microprocessor based devices such as a mobile phone, a smartphone, a tablet computer, an iPad®, an e-reader, or wearable computers such as smart watches, intelligent eyewear and the like.

For the embodiments of the invention as presented in this application using such a computer 7000, software code representing the equivalent of the prediction program, algorithms, and databases may be read from storage devices 7050 or 7051 within the computer system 7000, or from CRM such as an optical disk 7062 or USB thumbdrive 7063, and executed using the CPU 7001 and system memory 7005. Instructions for user input or final predicted results may be presented on either an internal display 7080 or an external display 7780 connected by means of an interface 7088, and the user may make “selections” using a keyboard 7090 and/or mouse 7092 synchronized with a graphical user interface (GUI) constructed within the software to allow coordination of the options shown on the available displays 7080 or 7780.

### VIII. Hardware and Software

Accordingly, embodiments of the present invention may be encoded in suitable hardware and/or in software (including firmware, resident software, microcode, etc.). Furthermore, embodiments of the present invention may take the form of a computer program product on a non-transitory computer readable storage medium having computer readable program code comprising instructions encoded in the medium for use by or in connection with an instruction execution system. Non-transitory computer readable media on which instructions are stored to execute the methods of the invention are therefore in turn embodiments of the invention as well. In the context of this application, a computer readable medium may be any medium that can contain, store, communicate, propagate, or transport the program for use by or in connection with the instruction execution system, apparatus, or device.

The computer readable medium may be, for example but not limited to, an electronic, magnetic, optical, electromagnetic, infrared, or semiconductor system, apparatus, or device. More specific examples (a non-exhaustive list) of a computer readable media would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM).

### IX. Additional Limitations

With this application, several embodiments of the invention, including the best mode contemplated by the inventors, have been disclosed. It will be recognized that, while specific embodiments may be presented, elements discussed in detail only for some embodiments may also be applied to others.

While specific materials, designs, configurations and fabrication steps have been set forth to describe this invention and the preferred embodiments, such descriptions are not intended to be limiting. Modifications and changes may be apparent to those skilled in the art, and it is intended that this invention be limited only by the scope of the appended claims.

We claim:

1. A computer implemented method for evaluating chemical compounds as potential pharmaceuticals, comprising:
importing data related to one or more selected chemical compounds;
determining, by a computer, based on the imported data,
one or more estimates of toxicity for the one or more chemical compounds;
exporting the determined estimates of toxicity; and
using the determined estimates of toxicity to specify a research protocol
for the evaluation of pharmaceutical efficacy and adverse effects
for at least one of the selected chemical compounds.

2. The computer implemented method of claim 1, in which
the data related to one or more selected chemical compounds
comprises gene expression data.

3. The computer implemented method of claim 1, in which
the data related to one or more selected chemical compounds
comprise transcript counts from quantitative polymerase chain reaction (qPCR).

4. The computer implemented method of claim 1, in which
the step of determining, by a computer, one or more estimates of toxicity
comprises the application of at least one selected algorithm;
and in which
the selection of the at least one algorithm
and the parameters used with the at least one algorithm
are determined using machine learning techniques.

5. The computer implemented method of claim 4, in which
the selection of the at least one algorithm and determination of the parameters using machine learning techniques is based on
data comprising predictors and also comprising corresponding toxicity results.

6. The computer implemented method of claim 1, in which
the research protocol comprises:
an evaluation of the toxicity of a chemical compound in preparation for a whole animal toxicity study.

7. The computer implemented method of claim 1 in which
the research protocol comprises:
synthesizing additional variations of the selected chemical compounds.

8. The computer implemented method of claim 1 in which
the research protocol comprises:
an evaluation of the structure of at least one of the selected chemical compounds.

9. The computer implemented method of claim 1 in which
the research protocol comprises:
an evaluation of physiological data.

10. A computer implemented method for evaluating chemical compounds, comprising:
importing data related to at least one selected chemical compound;
determining, by a computer, one or more estimates of toxicity
for the at least one selected chemical compound based on the imported data; and
exporting the determined estimates of toxicity.

11. The computer implemented method of claim 10, in which
the data related to at least one selected chemical compound
comprise microarray data.

12. The computer implemented method of claim 10, in which
the data related to at least one selected chemical compound
comprise gene expression data.

13. The computer implemented method of claim 10, in which
the data related to at least one selected chemical compound
comprise transcript counts from quantitative polymerase chain reaction (qPCR).

14. The computer implemented method of claim 10, in which
the data related to at least one selected chemical compound
comprise data previously made publicly available by an entity
selected from the group consisting of
the U.S. Food and Drug Administration,
the Japanese Toxicogenomics Project,
Entelos Inc., Iconix Biosciences and Johnson and Johnson.

15. The computer implemented method of claim 10, in which
the data related to at least one selected chemical compound
comprise data related to mammalian liver cells.

16. The computer implemented method of claim 15, in which
the mammals used as the source of the mammalian liver cells
are selected from the group consisting of
rats, dogs, cats, monkeys, apes and humans.

17. The computer implemented method of claim 15, in which
the liver cells are hepatocytes.

18. The computer implemented method of claim 15, in which
the hepatocytes are prepared from multiple individuals.

19. The computer implemented method of claim 10, in which
the step of determining one or more estimates of toxicity
uses a coefficient penalized linear regression algorithm.

20. The computer implemented method of claim 19, in which
the coefficient penalized linear regression algorithm comprises
an algorithm selected from the group consisting of
the Lasso Regression algorithm, the Ridge Regression algorithm, the ElasticNet algorithm and the glmnet algorithm.

21. The computer implemented method of claim 10, in which
the step of determining one or more estimates of toxicity
uses a binary decision tree algorithm.

22. The computer implemented method of claim 21, in which
the binary decision tree algorithm comprises
an algorithm selected from the group consisting of
the Bagging algorithm, the Random Forests algorithm, the Gradient Boosting algorithm and the Stochastic Gradient Boosting algorithm.

23. The computer implemented method of claim 10, in which
the step of determining one or more estimates of toxicity
uses a neural network method selected from the group consisting of:
the Restricted Boltzmann Machine method, the Feed-forward Neural Net method and the Deep Belief Networks method.

24. The computer implemented method of claim 10, in which
the step of determining one or more estimates of toxicity
uses a Support Vector Machine algorithm.

25. The computer implemented method of claim 10, in which
the step of determining one or more estimates of toxicity
additionally comprises:
making an estimation of a biological assay variable related to liver pathology.

26. The computer implemented method of claim 25, in which
the biological assay variable is related
to physiological data.

27. The computer implemented method of claim 25, in which
the biological assay variable is related
to an estimation of drug induced liver injury.

28. The computer implemented method of claim 25, in which
the biological assay variable is related to an estimate of
a specific pre-determined liver pathology.

29. The computer implemented method of claim 28, in which
the specific pre-determined liver pathology is selected from the group consisting of
hypertrophy, necrosis, microgranuloma, cellular change and cellular infiltration.

30. The computer implemented method of claim 25, in which
the biological assay variable is related to an estimate of
toxicity in an organ selected from the group consisting of:
the heart, the kidney, the nerves, the lungs, the blood vessels and the brain.

31. The computer implemented method of claim 25, in which
the biological assay variable is related to
an estimate of the probability that the toxicity for the at least one chemical compound will be greater for cancerous tissue than for healthy tissue.

32. The computer implemented method of claim 10, in which
the step of determining one or more estimates of toxicity
uses a toxicity model created using machine learning techniques.

33. The computer implemented method of claim 32, in which
the machine learning techniques used to create the toxicity model comprise:
importing data related to one or more selected chemical compounds,
in which the imported data comprises predictors and results;
dividing the imported data into a first dataset and a second dataset,
in which the first dataset and the second dataset comprise predictors and results;
selecting at least one algorithm to relate predictors and results;
calculating, by a computer, a set of parameters
for use with the selected at least one algorithm,
and in which said calculation is carried out
using the predictors and results of the first dataset; and then
computing a set of estimated results, based on
at least some of the predictors in the second dataset,
the selected at least one algorithm, and
the computed set of parameters; and
comparing the set of estimated results with the corresponding results of the second dataset.

34. The computer implemented method of claim 33, in which
the selected at least one algorithm is
a coefficient penalized linear regression algorithm.

35. The computer implemented method of claim 33, in which
the selected at least one algorithm is
a binary decision tree algorithm.

36. The computer implemented method of claim 33, in which
the step of dividing the imported data into a first dataset and a second dataset
comprises assigning any imported data related to any single chemical compound into the same dataset.

37. The computer implemented method of claim 33, in which
the imported data related to one or more selected chemical compounds
additionally comprises data related to dose.

38. The computer implemented method of claim 33, in which
the imported data related to one or more selected chemical compounds
additionally comprises data related to time of delivery.

39. The computer implemented method of claim 32, in which
the machine learning techniques used to create the toxicity model comprise:
importing data related to one or more selected chemical compounds,
in which the imported data comprise predictors and results;
dividing the imported data into a first dataset and a second dataset;
selecting at least two or more algorithms to relate predictors and results;
computing, for each of the selected algorithms, a set of parameters,
said computations carried out
using at least some of the predictors and results of the first dataset; and then
computing a set of estimated results, based on
at least some of the predictors in the second dataset;
the selected two or more algorithms, and
the sets of parameters for the selected algorithms; and
comparing the set of estimated results with the corresponding results of the second dataset.

40. The computer implemented method of claim 10, in which
the exported estimates of toxicity comprise
a description of the probability that
an adverse toxic effect will occur for the at least one chemical compound.

41. The computer implemented method of claim 10, in which
the exported estimates of toxicity comprise
an estimate of the probability
that the toxicity for the at least one chemical compound
will be greater for cancerous tissue than for healthy tissue.

42. The computer implemented method of claim 10, in which
the exported estimates of toxicity
are stored in a database.

43. A computer implemented method for evaluating chemical compounds, comprising:
importing gene expression data related to at least one selected chemical compound;
determining, by a computer, based on the imported data,
one or more estimates of toxicity for the at least one chemical compound; and
exporting the determined estimates of toxicity; and in which
said step of determining uses a toxicity model
created using machine learning techniques comprising:
importing data related to one or more selected chemical compounds,
in which the imported data comprises predictors and results;
dividing the imported data into a first dataset and a second dataset,
in which the first dataset and the second dataset comprise predictors and results;
selecting at least one algorithm to relate predictors and results;
calculating, by a computer, a set of parameters
for use with the selected at least one algorithm,
and in which said calculation is carried out
using the predictors and results of the first dataset; and then
computing a set of estimated results, based on
at least some of the predictors in the second dataset,
the selected at least one algorithm, and
the computed set of parameters; and
comparing the set of estimated results with the corresponding results of the second dataset;
and in which
the exported estimates of toxicity comprise
a description of the probability that
an adverse toxic effect will occur for the at least one chemical compound.
